SEQUENCE LISTING (2 sequences)

SEQ ID NO: 1 - protein, 39 aa, Homo sapiens
MENFQKVEKIGEGTYGVVYKARNKLTGEVVALKKIRSTA

SEQ ID NO: 2 - protein, 41 aa, Homo sapiens
DGRSLLSQMLHYDPNKRISAKAALAHPFFQDVTKPVPHLRL

(19) United States
(12) Patent Application Publication
(10) Pub. No.: US 2005/0214788 A1
(43) Pub. Date: Sep. 29, 2005

(54) Method and system for clustering and rescaling for molecular analysis

(75) Inventor: Jianwei Che, San Diego, CA (US)

Correspondence Address: QUINE INTELLECTUAL PROPERTY LAW GROUP, P.C., P O BOX 458, ALAMEDA, CA, 94501, US

(73) Assignee: IRM, LLC, Hamilton (BM)

(21) Appl. No.: 10/939,115

(22) Filed: Sep. 9, 2004

Related U.S. Application Data
(60) Provisional application No. 60/501,654, filed on Sep. 9, 2003.

(51) Int. Cl. C12Q 1/68; G01N 33/53; G06F 19/00; G01N 33/48; G01N 33/50

(52) U.S. Cl. 435/6; 435/7.1; 702/19

(57) ABSTRACT

Methods and/or systems for modeling molecular systems and/or other physical systems using scaling optimization.

### CROSS REFERENCE TO RELATED APPLICATIONS

This application claims priority from provisional patent application 60/501,654, filed 9 Sep. 2003 and incorporated herein by reference.

### COPYRIGHT NOTICE

Pursuant to 37 C.F.R. 1.71(e), Applicants note that a portion of this disclosure contains material that is subject to and for which is claimed copyright protection (such as, but not limited to, source code listings, screen shots, user interfaces, or user instructions, or any other aspects of this submission for which copyright protection is or may be available in any jurisdiction.). The copyright owner has no objection to the facsimile reproduction by anyone of the patent document or patent disclosure, as it appears in the Patent and Trademark Office patent file or records. All other rights are reserved, and all other reproduction, distribution, creation of derivative works based on the contents, public display, and public performance of the application or any part thereof are prohibited by applicable copyright law.

### COMPUTER PROGRAM LISTINGS APPENDIX COMPACT DISC

This application is being filed with a computer program listings appendix on compact disc, containing one file named Code_Listing.36-0042.10.9Sept04.doc. The computer program listings appendix on compact disc sets out selected source code extracts from a copyrighted software program, owned by the assignee of this patent document, which manifests the invention. This example source code is written in the C++ programming language and can be executed as modules in conjunction with a variety preconditioned conjugated gradient or conjugated gradient subroutines such as those available from Numerical Recipes (www.nr.com) and other available libraries. Permission is granted to make copies of the appendices solely in connection with the making of facsimile copies of this patent document in accordance with applicable law; all other rights are reserved, and all other reproduction, distribution, creation of derivative works based on the contents, public display, and public performance of the appendix or any part thereof are prohibited by the copyright laws. The contents of the computer program listings appendix on compact disc submitted with this application are incorporated herein by reference for all purposes.

These appendices and all other papers filed herewith, including papers filed in any attached Information Disclosure Statement (IDS), are incorporated herein by reference.

### FIELD OF THE INVENTION

The present invention relates to a method and/or system and/or apparatus for performing improved chemical modeling and/or other physical system modeling. In specific embodiments, the invention involves a method and/or system and/or apparatus for determining ligand conformations in receptor/ligand docking modeling. In further embodiments, the invention involves one or methods that may be implemented on a data handling device or system, such as a computer or other information enabled device. In further embodiments, the invention involves methods and/or systems for utilizing improved modeling over a communication network.

### BACKGROUND OF THE INVENTION

Despite significant progress made in past decades, the determination of conformational equilibriums of complex molecular systems still remains as one of the most difficult challenges in molecular modeling. Even using classical approximations, the potential energy surface describing the microscopic state of a molecular system is usually a complicated, multidimensional function of up to all atomic coordinates. The complexity and roughness of an energy surface are characterized by the large number of energy barriers separating important local minima. Moreover, the number of energy barriers generally increases exponentially with the size of the system.

This exponential increase is one of the major obstacles behind the ligand receptor docking problem and unsolved protein folding problems. Standard optimization methods for finding functional optima are often inefficient when applied to molecular potential functions.

One statement of the Receptor-Ligand Docking Problem is as follows: given a receptor and a potential ligand molecule, predict whether the molecule will bind to the receptor and, if so, the geometry and/or affinity of the binding. In other words, if the molecule is a ligand of the receptor, predict the relative orientation of the two molecules in their bound state. The geometry of the receptor-ligand complex is generally the configuration of the two that minimize free energy (e.g., potential energy+entropy). Docking predictions are important tools in many research and commercial settings, such as structure-based rational drug design, enzyme design, protein characterization studies, etc.

In general, there is a close fit between the two molecules, and in real-world events, the ligand and/or the receptor will deform to accommodate this tight fit. One application of receptor-ligand-docking estimations is to quickly screen large numbers of possible compounds that fit a particular receptor and select those that show a high affinity. Identified ligands can then be screened by other computational techniques or directly in assays.

Many methods have been proposed for predicting ligand-receptor interactions. Early techniques made simplifying assumptions, such as rigid ligands and a rigid protein. Other techniques, assume some flexibility for ligands and/or receptors. However, for large molecules and/or for complex molecular systems, the total conformational space is so large that it is very difficult to compute a structure and/or interaction and/or conformation corresponding to the global energy minimum.

Various computer-implemented methods have been proposed for searching the conformational space to identify structures that correspond to local energy minima. Some prior conformational search methods can be classified as systematic (deterministic) or stochastic (probabilistic). In systematic search procedures, such as a grid scan search, specified torsion angles are varied over a grid of equally spaced values. When torsion angles are varied systematically over their entire range, an overall picture of the energy landscape can be generated, the quality of which is dependent on the spacing of the grid. However, this method has been found to be impractical for more than about four torsion angles, which limits its use to small molecules.

Stochastic search procedures generally involve some type of random alterations to a structure. Common implementations include Monte Carlo methods and molecular dynamics. Monte Carlo methods, for example, can involve random changes in torsion angles as a way to sample different regions of conformational space. In molecular dynamics, the search involves sampling structures at intervals of time during a simulation. Simulated annealing is an application of molecular dynamics designed for conformational searching. In this process, structures are heated to temperatures at which increased atomic motion will occur in an attempt to drive molecules out of their local energy wells. Structures are then cooled slowly to trap them in the new local energy minimum.

Many modern docking algorithms make a simplifying assumption that the receptor is a rigid object. Receptor structures can be determined by x-ray crystallography or NMR spectroscopy. With this assumption, the degrees of spatial freedom of the problem generally are those of the ligand, which generally are three translational, three global-rotational, and one internal rotation for each rotatable bond. It is often assumed that bond lengths and the angles formed by adjacent bonds do not change, and on the scale of most ligands (10 to 80 atoms), this assumption is generally a reasonable one.

Methods for determining docking can be described in terms of a placement sampling algorithm to generate configurations of the ligand within the binding pocket of the receptor and a scoring function or direct energy function to rank placements for iteration or final results.

Although each placement could be completely random and independent, most algorithms either use heuristics based on the chemistry or geometry of the atoms involved (e.g., the FlexX and DOCK methods), or use a standard optimization technique such as simulated annealing or a genetic algorithm (such as Autodock). Some use explicit molecular dynamics simulation.

Examples of general types of scoring functions include explicit force field, empirical and knowledge-based.

A number of approaches have been discussed based on clustering. One example can be found in Charles D. Schwieters and G. Marius Clorey, Internal Coordinates for Molecular Dynamics and Minimization in Structure Determination and Refinement, Journal of Magnetic Resonance 152, 288-302 (2001). Other references that provide various background information are listed below.

The discussion of any work, publications, sales, or activity anywhere in this submission, including in any documents submitted with this application, shall not be taken as an admission that any such work constitutes prior art. The discussion of any activity, work, or publication herein is not an admission that such activity, work, or publication existed or was known in any particular jurisdiction.

References

1. A Survey of Methods for Searching the Conformational Space of Small and Medium sized Molecules, pages 1-55, Reviews in Computational Chemistry, VCH publishers, New York, 1991
2. J. Blaney and J. Dixon, Perspectives in Drug Discovery and Design 1, 301 (1993)
3. Searching Conformational Space, vol. 2 of Computer Simulation of Biomolecular Systems, ESCOM Science Publishers, Leiden
4. J. Klepeis and C. Floudas, J. Chme. Phys. 110, 7491 (1999)
5. K. Gibson and H. Scheraga, J. Comp. Chem. 11, 468 (1990)
6. A. Mazur, V. Dorofeev, and R. Abagyan, J. Comp. Phys. 92, 261 (1991)
7. A. Jain, N. Vaidehi, and G. Rodriguez, J. Comp. Phys. 106, 258 (1993)
8. Charles D. Schwieters and G. Marius Clorey, Internal Coordinates for Molecular Dynamics and Minimization in Structure Determination and Refinement, Journal of Magnetic Resonance 152, 288-302 (2001).
9. Morris G. M., Goodsell D. S., Halliday R. S., Huey R., Hart W. E., Belew R. K. and Olson A. J., J Comp Chem, 19 (14):1639-1662, 1998.
10. VanGunsteren W. F. and Berendsen H. J. C., “Groningen Molecular Simulation (GROMOS) Library Manual”, Biomos, Groningen.
11. Smith L. J., Mark A. E., Dobson C. M. and van Gunsteren W. F., Biochemistry, 34:10918-10931, 1995.
12. Pascutti P. G., Mundim K. C., Ito A. S. and Bisch P. M., J Comp Chem, 20 (9):971-982, 1999.
13. Arora N. and Jayaram B., J Comp Chem, 18 (9):1245, 1997.
14. Eldridge, M. D., C. W. Murray, T. R. Auton, G. V. Paolini, and R. P. Mee. Empirical Scoring Functions. I. The Development of a Fast, Fully Empirical Scoring Function to Estimate the Binding Affinity of Ligands in Receptor Complexes. Journal of Computer-Aided Molecular Design, 1997. 11:425-445.
15. Boehm, H-J. The Development of a Simple Empirical Scoring Function to Estimate the Binding Constant for a Protein-Ligand Complex of Known Three-Dimensional Structure. Journal of Computer-Aided Molecular Design, 1994. 8:243-256.
16. Muegge, I. and Y. C. Martin. A General and Fast Scoring Function for Protein-Ligand Interactions: A Simplified Potential Approach. Journal of Medicinal Chemistry, 1999. 42(5):791-804.
17. Gohlke, H., M. Hendlich, and G. Klebe. Knowledge Based Scoring Function to Predict Protein-Ligand Interactions. Journal of Molecular Biology, 2000. 295:337-356.
18. Morris, G. M., et al., Automated docking using a Lamarckian genetic algorithm and an empirical binding free energy function. Journal of Computational Chemistry, 1998. 19(14): p. 1639-1662.
19. Kramer, B., M. Rarey, and T. Lengauer, Evaluation of the FLEXX incremental construction algorithm for protein-ligand docking. Proteins, 1999. 37(2): p. 228-41.
20. Rarey, M., S. Wefing, and T. Lengauer. Placement of Medium-Sized Molecular Fragments Into Active Sites of Proteins. Journal of Computer-Aided Molecular Design, 1996. 10:41-54.
21. Kuntz, I. D., J. M. Blaney, S. J. Oatley, R. Langridge, and T. E. Ferrin. A Geometric Approach to Macromolecule-Ligand Interactions. Journal of Molecular Biology, 1982. 161:269-288.
22. Ewing, T. J. A. and I. D. Kuntz, Critical evaluation of search algorithms for automated molecular docking and database screening. Journal of Computational Chemistry, 1997. 18: p. 1175-1189.

### SUMMARY

The present invention, in specific embodiment, involves a novel and efficient approach for finding the best conformations of ligand molecules inside a receptor binding site for predicting receptor-ligand complex 3-d structures. In other embodiments, methods of the invention can be applied to related problems, such as macromolecular modeling.

Methods of the invention according to specific embodiments involve representing a ligand molecule by topological clusters and rescaling the conformational and the orientational degrees of freedom of the ligand iteratively while applying one or more standard optimization and/or scoring and/or energy determination functions. According to specific embodiments, the invention essentially searches the rough energy landscape inside the binding site (e.g., by changing the gradient in a gradient based optimization function) to find a much smoother path that allows the ligand to reach its better optimal conformation more effectively and provides improvements in finding global optimal conformation. Even though ligand molecules of interests may be of small to medium size, the ligand receptor binding complexes are complicated molecular systems.

The present invention according to specific embodiments involves an algorithm that takes into account the molecular geometric nature of the potential energy function and improves the optimization effectiveness significantly.

The potential energy surface that a ligand molecule “feels” is rendered by macromolecular receptors. In most case, the energy surface inside the binding site is much more rough and complicated than the one of a free ligand molecule, therefore, the traditional methods for obtaining small molecular 3D optimal structures in vacuum or gas phase, such as standard conjugate gradient, preconditioned conjugated gradient, simulated annealing, steepest descend, are no longer effective. While these methods are at times effective for optimizing smooth and simple functions, the methods often get trapped in local minimums very easily, and become not very useful for predicting accurate ligand receptor binding structures.

The present invention in specific embodiments can take advantage of the best features in traditional optimization methods, but more importantly considers the characteristics of ligand binding inside the receptor. Consequently, it greatly improves the convergence. According to specific embodiments of the invention, the invention accomplishes this by scaling a gradient used to measure the energy surface or one or more parameters used to sample ligand conformational space more efficiently.

By representing a ligand molecule as a set of hierarchical clusters and rescaling the gradients or movements consistently according to cluster size, the present invention achieves a significant improvement in the convergence during energy minimization for ligand molecules under the influence of an external field, such as in a receptor binding site. This is particularly useful for docking ligand molecules into receptors when tens of thousands or more of conformations for each ligand molecule need to be evaluated.

The invention according to specific embodiments may be further applied in some optimization algorithms where no gradients are used during optimization such as Monte Carlo method. In this circumstance, the scaling is applied to transformation of atom movements directly. However, in gradient based optimization, generally a gradient is provided so that the optimization can “know” locally what the slope is and thereby adjust some aspects of the optimization. Generally, a gradient for a given function is directly calculated from the function. According to specific embodiments of the invention, the invention scales the gradient according to the size of the cluster and directs an optimization algorithm to use the new gradient.

Software Implementations

Various embodiments of the present invention provide methods and/or systems for molecular modeling that can be implemented on a general purpose or special purpose information handling appliance using a suitable programming language such as Java, C++, C#, Perl, Cobol, C, Pascal, Fortran, PL1, LISP, assembly, etc., and any suitable data or formatting specifications, such as HTML, XML, dHTML, tab-delimited text, binary, etc. In the interest of clarity, not all features of an actual implementation are described in this specification. It will be understood that in the development of any such actual implementation (as in any software development project), numerous implementation-specific decisions must be made to achieve the developers' specific goals and subgoals, such as compliance with system-related and/or business-related constraints, which will vary from one implementation to another. Moreover, it will be appreciated that such a development effort might be complex and time-consuming, but would nevertheless be a routine undertaking of software engineering for those of ordinary skill having the benefit of this disclosure.

Other Features & Benefits

The invention and various specific aspects and embodiments will be better understood with reference to the following drawings and detailed descriptions. For purposes of clarity, this discussion refers to devices, methods, and concepts in terms of specific examples. However, the invention and aspects thereof may have applications to a variety of types of devices and systems. It is therefore intended that the invention not be limited except as provided in the attached claims and equivalents.

Furthermore, it is well known in the art that logic systems and methods such as described herein can include a variety of different components and different functions in a modular fashion. Different embodiments of the invention can include different mixtures of elements and functions and may group various functions as parts of various elements. For purposes of clarity, the invention is described in terms of systems that include many different innovative components and innovative combinations of innovative components and known components. No inference should be taken to limit the invention to combinations containing all of the innovative components listed in any illustrative embodiment in this specification.

All references, publications, patents, and patent applications cited herein are hereby incorporated by reference in their entirety for all purposes.

### BRIEF DESCRIPTION OF THE DRAWINGS

FIG. 1 shows portions of an example initial ligand data file that can be used in a computer implemented method according to specific embodiments of the invention.

FIG. 2 shows portions of an example final state data file (in this example, for a receptor binding site) that can be used in a computer implemented method according to specific embodiments of the invention.

FIG. 3(a) and (b) illustrate representations of a ligand molecule for 1G5S by clusters.

FIG. 4 provides a schematic illustration of the variation of the radius of gyration of the parent cluster upon rotation of its child cluster (here the benzene ring) according to specific embodiments of the invention.

FIG. 5 provides schematic illustration of five ligand molecules (in co-crystals) under study: (a) 1G5S, (b) 1DI9, (c) 1BL7, (d) 1FVV and (e) 1KV1 having 7, 8, 4, 5 and 10 child clusters respectively.

FIG. 6A provides a schematic illustration of ligand 1FVV showing (a) initial conformation and crystal state (b) converged states obtained from ICR (internal coordinate representation) with scaling and ICR without scaling.

FIG. 6B provides a schematic illustration of ligand 1DI9 (a) initial conformation and crystal state (b) converged states obtained from rescaling and all atom optimization.

FIG. 7 is an example flowchart showing an overall method for ligand docking determination that can be implemented on an information system according to specific embodiments of the invention.

FIG. 8 shows portions of an example output structural file using rescaling optimizations from a computer implemented method according to specific embodiments of the invention.

FIG. 9A-F shows portions of an example processing log file using rescaling optimizations from a computer implemented method according to specific embodiments of the invention.

FIG. 10A-D shows portions of an example processing log file using a non-rescaling optimizations from a computer implemented method according to specific embodiments of the invention.

FIG. 11 is a block diagram showing a representative example logic device in which various aspects of the present invention may be embodied.

### DESCRIPTION OF SPECIFIC EMBODIMENTS

Before describing the present invention in detail, it is to be understood that this invention is not limited to particular compositions or systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content and context clearly dictates otherwise. Thus, for example, reference to “a device” includes a combination of two or more such devices, and the like.

Unless defined otherwise, technical and scientific terms used herein have meanings as commonly understood by one of ordinary skill in the art to which the invention pertains. Although any methods and materials similar or equivalent to those described herein can be used in practice or for testing of the present invention, the preferred materials and methods are described herein.

1. Overview

The present invention according to specific embodiments can be implemented in a computer system, laboratory system, or other information handling device.

The invention may be better understood by first considering usual steps in a prior art conformation determination or docking problem. These steps may be understood as follows:
1. Determine an initial conformation of a molecular system to be optimized (e.g., an initial conformation of a ligand.)
2. Determine the parameters or environment of a final desired state (e.g., the energy characteristics and/or geometry of a receptor binding site)
3. Determine a number of various possible conformations for the molecular system to be optimized.
4. Filter and optimize each conformation obtained from step 3 according to the property of molecular system (e.g. optimum energy characteristics).
5. Score the optimized conformations and select the best one or a few conformations as the final conformation prediction(s).

While these general steps can apply to a variety of different chemical modeling situations, such as protein folding, etc., further details here will reference a ligand/receptor docking problem.

Initial Data Regarding Molecular System to be Optimized

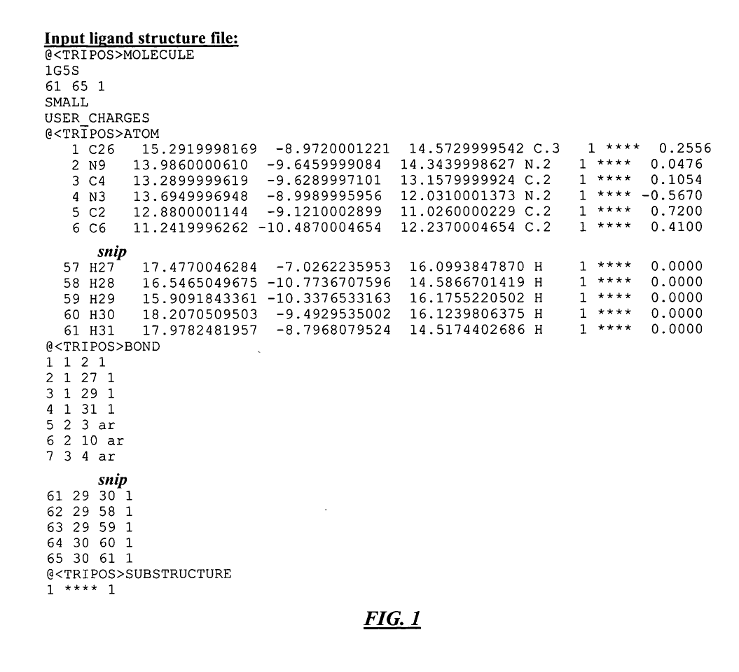

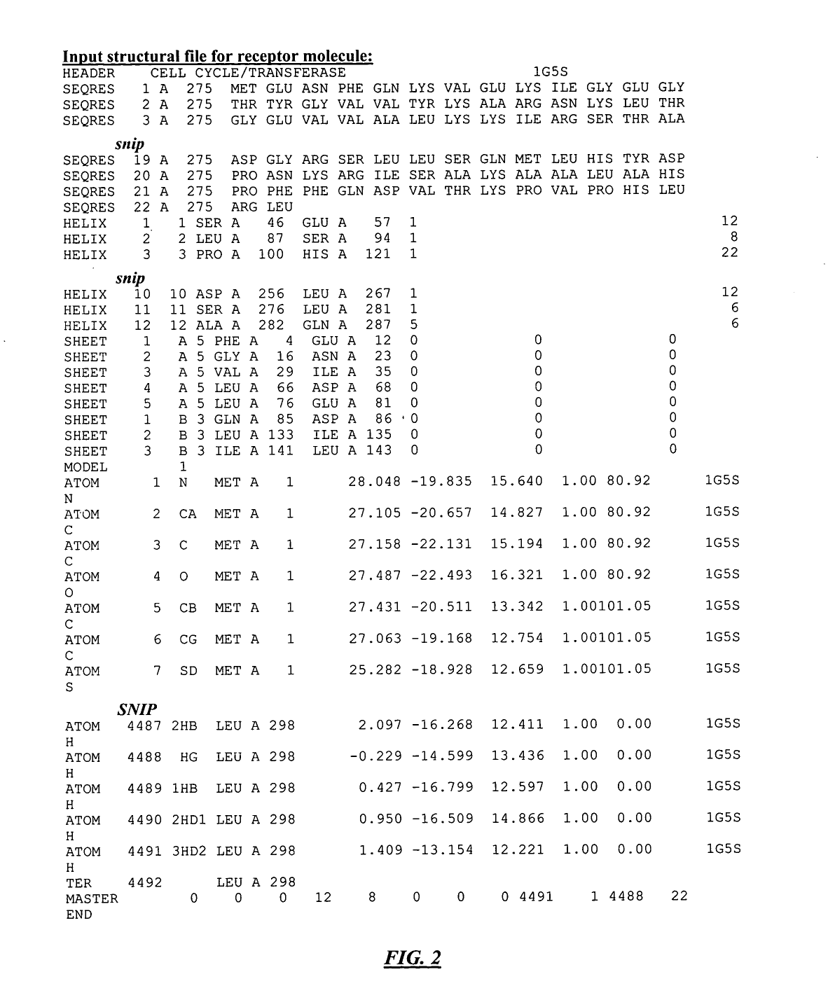

An example initial conformation is the isolated structure of a particular candidate ligand and its corresponding receptor of interest. For computer implemented modeling, such a structure is generally specified in one or more numerical data files that indicate atoms or other structures of a candidate ligand and receptor and geometric positions of the atoms or other structures. FIG. 1 shows portions of an example initial ligand data file that can be used in a computer implemented method according to specific embodiments of the invention. This file generally is supplied in a standardized format, such as PDB or MOL2.

Initial data can be determined in a variety of ways, such as X-ray crystallography, homology modeling, and/or computational prediction.

Parameters for a Final State

In a ligand docking application, the parameters for a final state may include the geometry and/or energy data of a binding site of a target receptor and bound ligand geometry, and may include a rigid model for a receptor or a model allowing some flexibility in a receptor binding site. FIG. 2 shows portions of an example final state data file (in this example, for a receptor binding site) that can be used in a computer implemented method according to specific embodiments of the invention.

Various Possible Conformations

For a potential ligand of fixed chemical composition, various possible conformations can include up to all possible geometric arrangements of constituent parts and/or atoms. Generally, some selection must be done to exclude a large set of unlikely and/or impossible conformations before conformations are tested. According to specific embodiments of the invention, a clustering algorithm as will be understood in the art is applied to a potential ligand to reduce the total number of considered possible conformation, since bond lengths and angles are fixed quantities in clusters.

To characterize a ligand structure inside a receptor binding site, 3N degrees of freedom are generally needed (if there is no symmetry involved) where N is the number of atoms in the ligand molecule or the number of bonds or other parameters allowed to be adjusted. Six degrees of freedom describe the position and orientation of the ligand as a whole in a receptor docking problem. The rest of them describe the detail geometric arrangement of each individual atom or moveable parts of the ligand.

How ligand atoms arrange themselves in a binding site is generally determined by the interaction field with their environment and between them. Within the classical approximation, the interaction field between atoms is a mathematical function of their 3 dimensional coordinates in space. In a docking problem, therefore, searching for an optimal (e.g., a lowest energy) structure therefore becomes the problem of finding optima of a mathematical function that depends on the position of up to all ligand and receptor atoms. In turn, the number of computationally stable binding conformations of a ligand can also be large.

Scoring Possible Conformations

Once one or more possible conformations are determined, it must be evaluated in some way to determine its desirability for example as compared to a final conformation prediction. In specific applications, scoring can also be referred to as energy ranking and can include determining the total energy of a particular conformation bound in a receptor. A variety of different energy calculation and/or scoring functions that are known in the art can be used according to specific embodiments of the invention.

In order to avoid getting a false final binding conformation, it is important to search the potential energy surface as effectively and thoroughly as possible, and to employ an optimization method that can avoid shallow nearby local minimums. The present invention addresses both aspect the problem and therefore improves the accuracy and efficiency for predicting ligand binding conformation in a receptor binding site.

2. Representing a Molecular System by Clusters

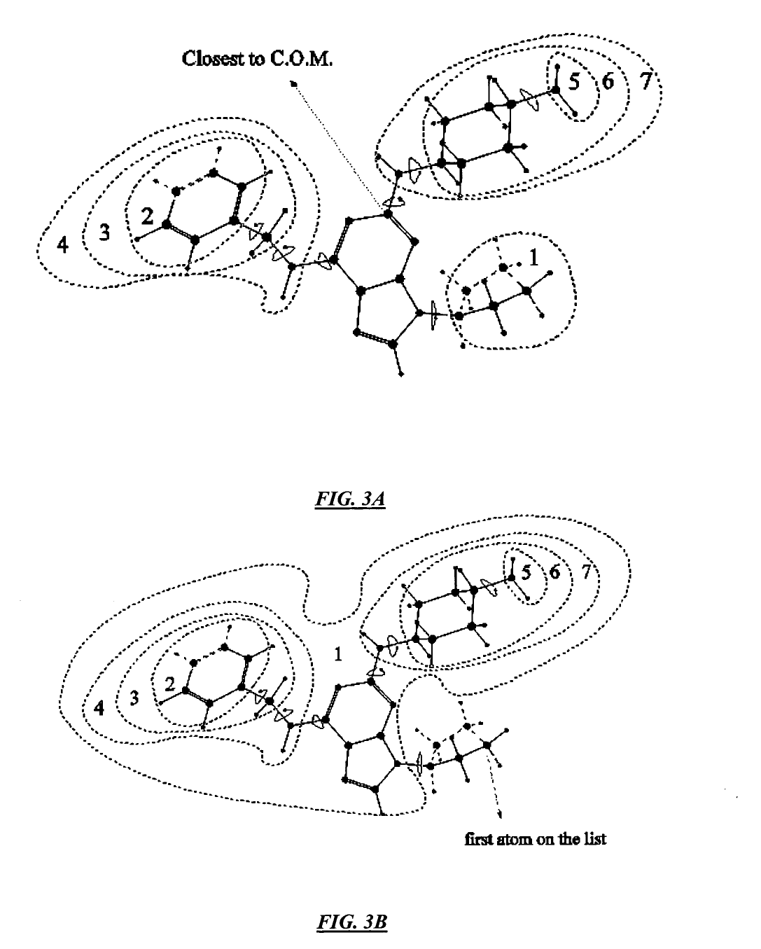

The method first represents a ligand molecule as a topological tree of clusters, as can be further understood with reference to references 5 and 6. Each cluster consists of child clusters that can rotate on a bond as described herein and/or a set of atoms whose relative positions among them are fixed. A few examples of molecular clustering are shown in FIG. 3. At various points of analysis each cluster can be treated as a rigid body formed by a collection of atoms. The criterion for forming a cluster is that the chemical bonds between the atoms in each cluster are not rotatable, i.e. these bonds are either double bonds, triple bonds, aromatic bonds, or a bond that belongs to a ring structure. Rotatable bonds are generally single bonds that are not part of a ring structure. These rotatable bonds can also be referred to as hinges connecting the clusters. The definition of rotatable bond can vary in different applications. For example, in specific cases some double bounds can be allowed to rotate but generally with a very high energy barrier, in other cases it may be desired to let a ring have some flexibility and therefore allow one or more ring bonds to rotate somewhat. Changing the rotatable bound definitions, however, does not require change to later steps of the algorithm.

As shown in FIG. 3, there is a hierarchical structure among the clusters where each cluster can either be a parent and/or a child of another cluster. According to specific embodiments of the invention, the entire ligand molecule itself is considered as a root cluster that can rotate and translate as a whole.

Clusters can be determined according to different methods and/or algorithms known in the art and/or according to proprietary methods. These methods are generally implemented on an information processing device. In general, clusterization starts with either (a) the atom closest to the center of mass of a ligand or (b) a randomly chosen atom that happens to be near the tip. Generally, the child clusters and the hierarchy among them are determined by a rule such as: a starting atom (non hydrogen) on the ligand molecule is selected as a root atom that is generally the closest to the center of mass, the next step is to determine neighboring atoms attached by rotatable bonds such that the rotation of such bonds would move a section of the ligand at one side of the bond with respect to the other side. In some embodiments, it is only designated a child cluster if the movement causes a change in the conformation without disrupting the chemical structure of the candidate ligand.

The atom on the side of the bond that belongs to the new child cluster can be referred to as the base cluster atom and the other atom is the parent cluster atom. Thus, the child cluster can also be said to be connected to the fixed framework by a hinge passing through the bond connecting the parent and the child cluster atoms.

In previous works, it was found computationally slightly more advantageous to start clusterizing with an atom somewhat mid-centered than those close to the tip of the molecule. FIG. 3b illustrates the same molecule with a different set of clusters formed when the starting point is near the tips. The major difference between the two cases is in the size of the clusters and the type of framework of the molecule to which the clusters are attached.

According to specific embodiments of the invention, the permissible motions of the ligand molecule during the conformational search are the translation and rotation of the entire ligand molecule as a whole, and the conformational flexibility of the molecule achieved by rotating each cluster around its own hinge axis.

3. Scoring Different Conformations

In the present invention, the size influence of each individual cluster is considered in the optimization. When a ligand molecule is placed inside a binding site, the rotations of each substructure are more or less restricted by the presence of receptor atoms. The restriction is more severe for tip atoms of a large cluster than central atoms of a small cluster. Because of the presence of the receptor, the direct optimization of interaction function becomes non-effective. Thus, the present invention employs a rescaling factor that scales each cluster movement or the energy gradient by its characteristic size (i.e. radius of gyration about the hinge axis). In order to take advantage of the existing efficient features in other optimization algorithms, according to specific embodiments of the invention, the invention uses a mathematically consistent transformation between scaled rotational degrees of freedoms and un-scaled ones along with corresponding gradients.

Illustration of Scaling Method

To illustrate the process, consider one cluster. First, the invention calculates the torque on this cluster about its hinge axis based on the interaction function with all the atoms in other clusters or molecules. The torque is the force needed to rotate the object of the cluster in a modeled energy field. This calculation according to specific embodiments of the invention is done outside of the optimization routine.

Second, the invention determines the radius of gyration (R_{g}) of the cluster, and scales the torque by the radius of gyration. Initially, this is done prior to the first optimization. The radius of gyration of a cluster can be calculated from its moment of inertia (I) using the relationship I=M R_{g}² where M is the total mass of the cluster. The moment of inertia about a given axis can be obtained from its inertia tensor as in the following, I=n⁺In where n is the unit vector in the direction of the rotation axis, n⁺ is the transpose of n, and I is the inertia tensor centered at the cluster base atom.

After performing this for all clusters, the scaled torques can be used as input to an optimization procedure, such as a gradient based optimization procedure. According to specific embodiments of the invention, because the scaling is done externally to the optimization process, existing and/or future optimization routines can be used generally without modification.

Third, a typical optimization method is carried out, and each cluster is rotated according to the rotation angle calculated from the optimization method.

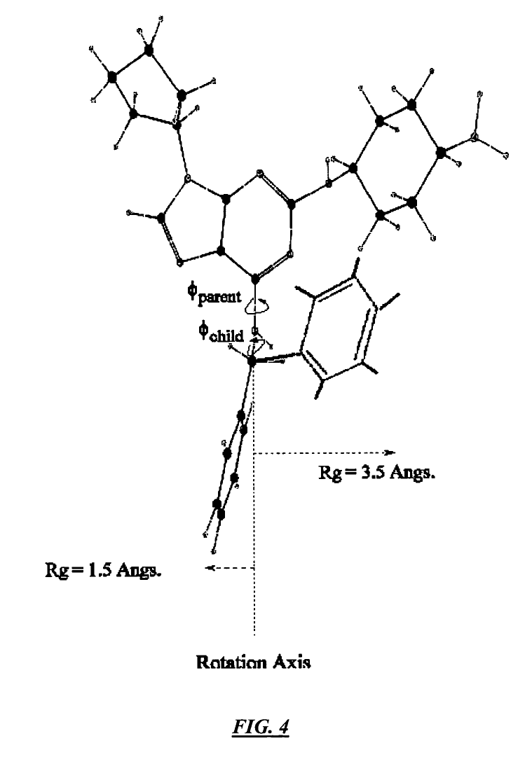

An important aspect of the algorithm is the variation of the radius of gyration of a cluster when one of its child clusters changes position upon rotation. This is illustrated in FIG. 4 where the change in the position of the benzene ring due to a rotation by an angle (Φ_{child} approximately doubles the size of the parent cluster to which it is attached. Therefore, it is important to update the radius of gyration consistently according to the optimization method applied. For example, the update is done for each new conformation accepted at the end of each iteration for steepest descent method, and only at restarting iteration for conjugate gradient method.

With the resealing algorithm, many harsh steric clashes between receptor atoms and ligand tip atoms can be avoided while still maintaining good chances for ligand to sample other areas of the potential energy surfaces. Consequently, it improves the efficiency for finding accurate ligand binding conformation.

It is generally understood that in a regular optimization algorithm, the output step size of the algorithm is based on the gradient. Large gradient indicates smaller step size. Smaller cluster with smaller torque moves very little. With a very harsh clash, however, although it is possible for a smaller cluster to get out of the bad conformations it does not allow the small cluster to move so much. Using the techniques of the present invention, the molecule moves in such a way that harsh steric clashes are avoided. Regular optimization do not employ this technique and thus do not get out of shallow wells as easily as specific embodiments of the current invention.

The invention may be better understood by first considering usual steps in a prior art conformation determination or docking problem. These steps may be understood as follows:
1. Determine an initial conformation of a molecular system to be optimized (e.g., an initial conformation of a ligand.)
2. Determine the parameters or environment of a final desired state (e.g., the energy characteristics and/or geometry of a receptor binding site)
3. Determine a number of various possible conformations for the molecular system to be optimized
4. Filter and optimize each conformation obtained from step 3 according to the property of molecular system (e.g. optimum energy characteristics).
5. Score the optimized conformations and select the best conformation one or a few as the final conformation prediction.

According to specific embodiments of the invention, after a ligand molecule is clusterized, it is placed in the energy field created by a receptor molecule and subject to further optimization in order to find the best binding conformation. In general, the number of conformations that a ligand molecule can adopt depends on its chemical nature. Among all conformations, the chemical bond lengths and bond angles usually do not alter appreciably for non-covalent binding, because the energy constraints on them are very stiff. However, the rotation about a single bond that is not a member of ring structure can be quite easy. In fact, different conformations are obtained by the rotation of these bonds. These conformations can be further optimized also by the rotation of these bonds to find the values giving rise to lowest energy. Thus, this is an optimization problem. The variables are the torsional angles between connected clusters. The rotation the clusters about is hinge axis (rotating bonds) is driven by the torque due to the energy field.

A torque of a cluster is calculated by the gradient of the energy field with respect to its rotation angle, for example as provided by the equation: Tᵢ(θ)=−∇_{θ}U, where i denotes the i^{th} cluster, θ is the rotation angle of the cluster about its hinge axis, U is the energy field which normally is a function of coordinates of atoms in the ligand molecule, and ∇_{θ} is the gradient of the energy with respect to the angle. Note that θ is a vector here, its amplitude is the value of the rotational angle, and its direction follows the right hand convention, i.e. the right hand fingers follow the direction of rotation, then the direction of the rotation vector is where the thumb points.

According to specific embodiments of the invention, the torque for each cluster is scaled by its radius of gyration before passing into normal gradient based optimization routines as gradient (e.g., Tᵢ^{new}(θᵢ)=Tᵢ(θᵢ)/Rᵢ, where Rᵢ is the radius of gyration of the i^{th} cluster). The regular optimization routine will search for the optimal step sizes (e.g., incremental rotation angles in this case).

### EXAMPLES

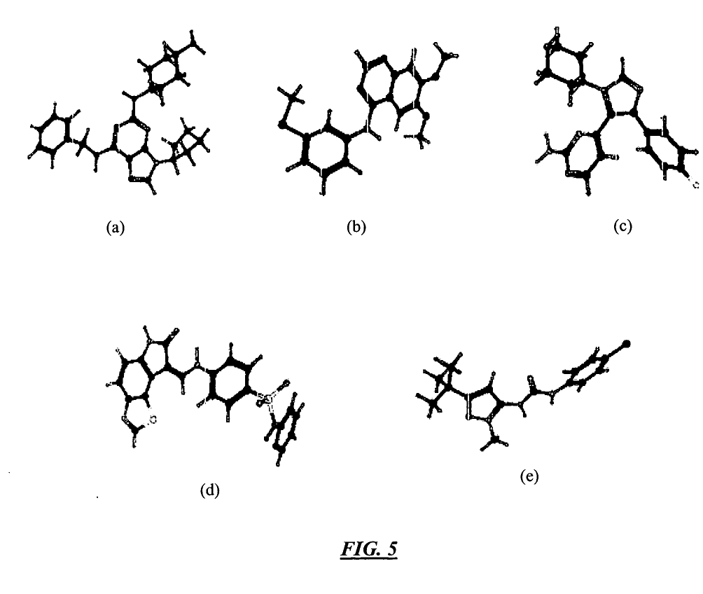

As examples further illustrating embodiments of the invention, one implementation of an algorithm according to specific embodiments of the invention was tested on a set of experimentally known structures of ligand/receptor complexes. FIG. 5 contains the structures of five ligand molecules as examples. For each ligand, starting conformations were generated by assigning randomly selected torsion angles to each rotatable bond while the bond lengths and bond angles remained fixed at their crystal state in the protein-ligand complex. Every generated conformation is then subjected to truncated Newton-Raphson energy minimization to derive the associated lowest energy conformation. The allowable set of motions during minimization consists of rotation of single bonds connecting the child clusters with their parent clusters, and the rotation and translation of the ligand molecule as a whole. The rotation of the whole ligand molecule is around its principal axes with the origin located at the center of mass.

TABLE 1

| | Rescaling Optimization | | | Nonscaling Optimization | | | |
|---|---|---|---|---|---|---|---|
| | E_{final} | | Time | E_{final} | | Time | |
| Samples | (kcal/mol) | iterations | (s) | (kcal/mol) | iterations | (s) | RMSD(Å²) |
| 1BL7 | −469.4 | 15 | 0.4 | −469.4 | 48 | 0.65 | 9.7e−5 |
| 1DI9 | −183.5 | 16 | 0.4 | −116.9 | 80 | 0.6 | 0.5 |
| 1FVV | −595.9 | 38 | 0.6 | −347.3 | 175 | 0.65 | 1.9 |
| 1G5S | −541.6 | 37 | 0.5 | −389.9 | 21 | 0.95 | 0.8 |
| 1KV1 | −481.6 | 41 | 0.3 | −481.6 | 113 | 0.6 | 7.e−4 |

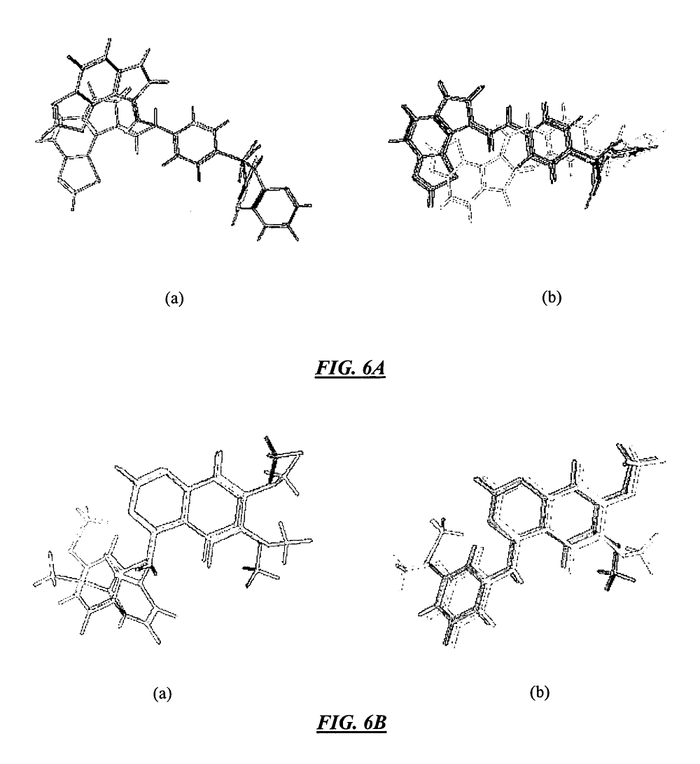

Table 1 provides a comparison between the optimization results obtained from scaling method and nonscaling cluster optimization for five different ligands. Table 1 shows the conformational energy of the converged final state of each ligand molecule inside its receptor binding site. The value of the total energy consists of the interaction energy between the protein and the ligand as well as the internal conformational energy of the ligand molecule (torsional, electrostatic, and dispersion energies). The convergence was reached once the root mean-square gradient was smaller than 0.01 kJ/mol/Å². It can be seen clearly that the optimization results obtained using resealing approach are better converged with less number of iterations in most cases and significantly shorter time intervals. Ligands 1BL7 , 1G5S, 1KV1 and 1DI9 are good examples that show the time difference since they all converged from the same initial state to the same final state based on their RMSD values. For ligand 1FVV, the minimum state reached by scaling optimization was much closer to crystal state than the minimum state reached without scaling which is well illustrated in FIG. 6A.

Comparing with traditional all atom optimization widely used in molecular modeling, the use of resealing cluster optimization is even more advantages. As with some prior nonscaling cluster based algorithms, the present invention makes it much easier to escape from nearby local minimums.

As illustrated in FIG. 6B, even if ligand 1DI9 was placed very close to the globally optimum location, all atom optimization was still unable to recover the best structure because it was trapped in a nearby local optimum. On the other hand, the scaling clustering optimization quickly found the global optimum, and it took less than 10 iterations to converge. Excellent agreement exists between this result and crystal state with an RMSD of 0.06.

TABLE 2

| Structures | Rescaling Optimization | Nonscaling Optimization |
|---|---|---|
| 1G5S | 5 | 2 |
| 1E1X | 11 | 6 |
| 1FVV | 3 | 0 |
| 1KV2 | 4 | 3 |

Table 2 provides a comparison of the frequency of finding the global optimal structure between the scaling and nonscaling optimizations. The results are collected from 300 random initial conditions. It can be seen clearly that the scaling approach significantly improves the chance of finding the correct binding conformation (i.e. the global optimum).

Example Flowchart

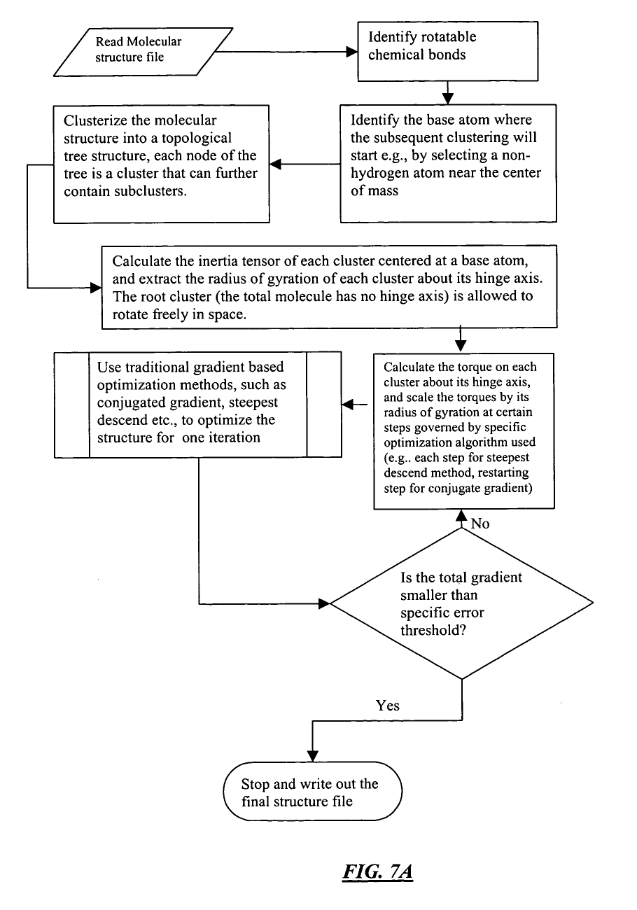

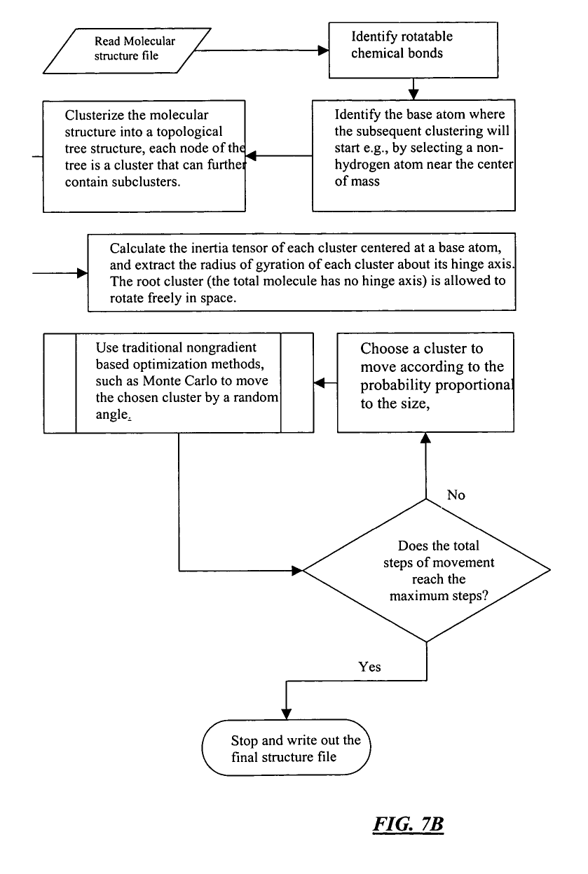

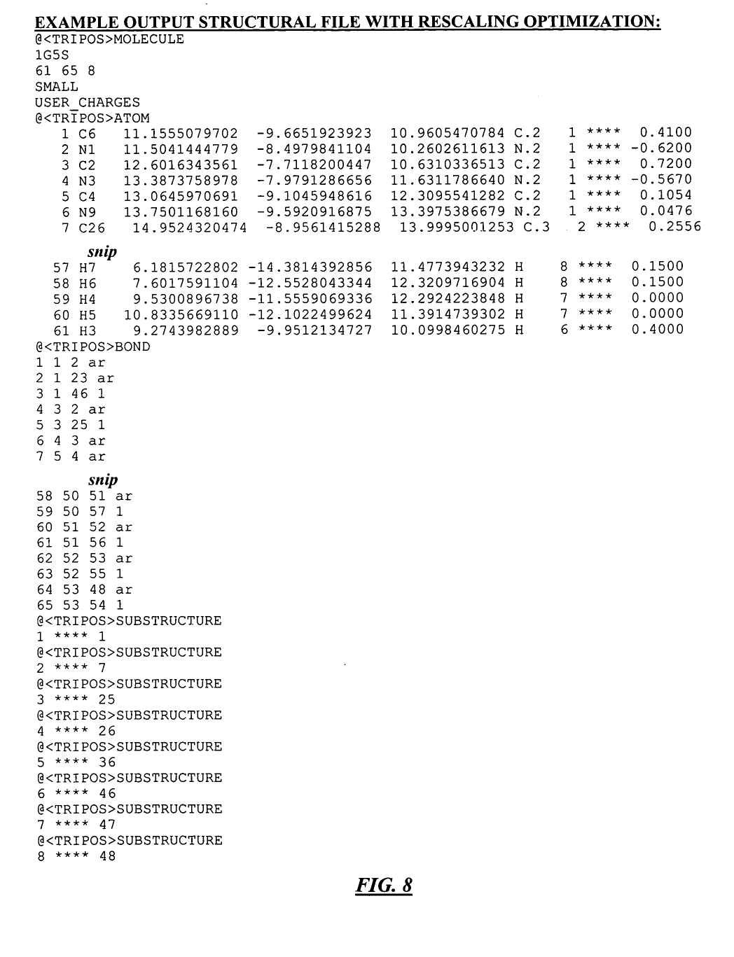

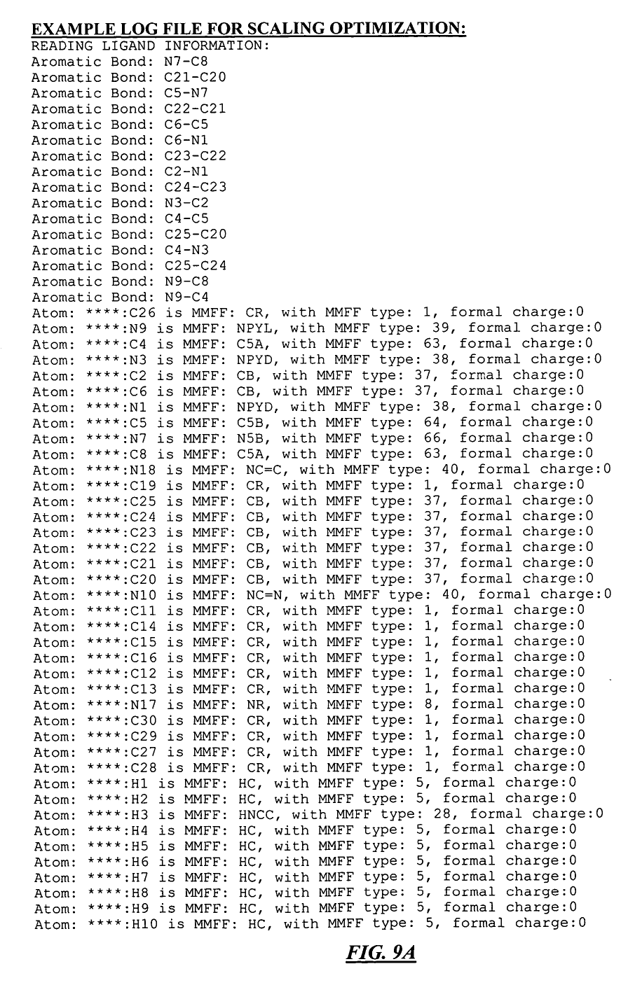

FIG. 7 is an example flowchart showing an overall method for ligand docking determination that can be implemented on an information system according to specific embodiments of the invention. Various specific implementations of such a method on a computer system can be performed according to specific embodiments of the invention as will be understood from the description herein to those of skill in the art. As described above, example input data files are provided for such a method in FIG. 1 and FIG. 2

FIG. 8 shows portions of an example output structural file using rescaling optimizations from a computer implemented method according to specific embodiments of the invention. This particular example file is presented in a standard format as will be understood in the art.

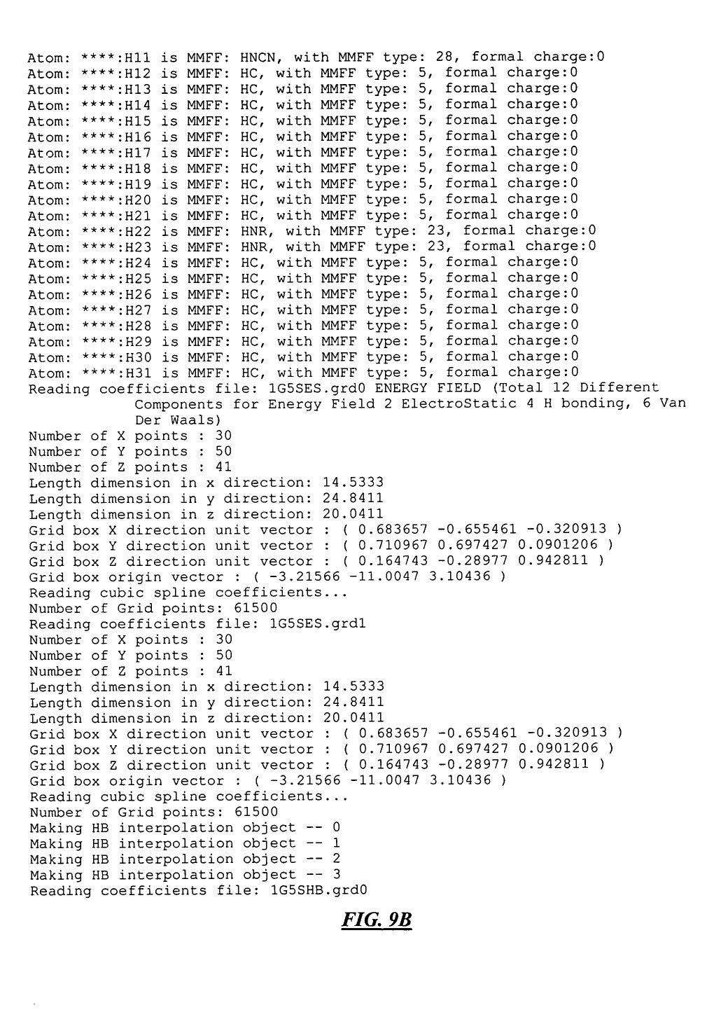

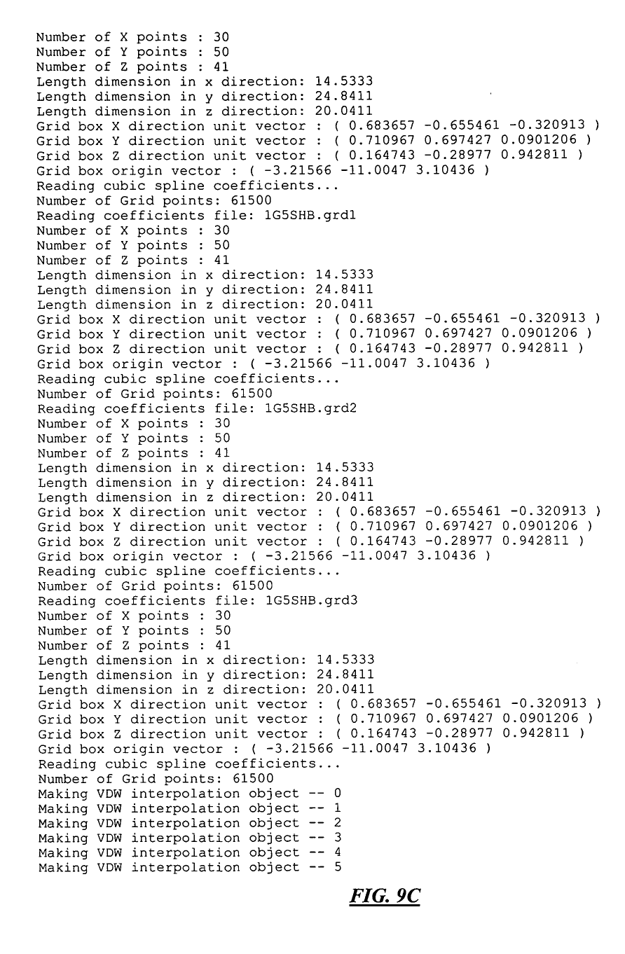

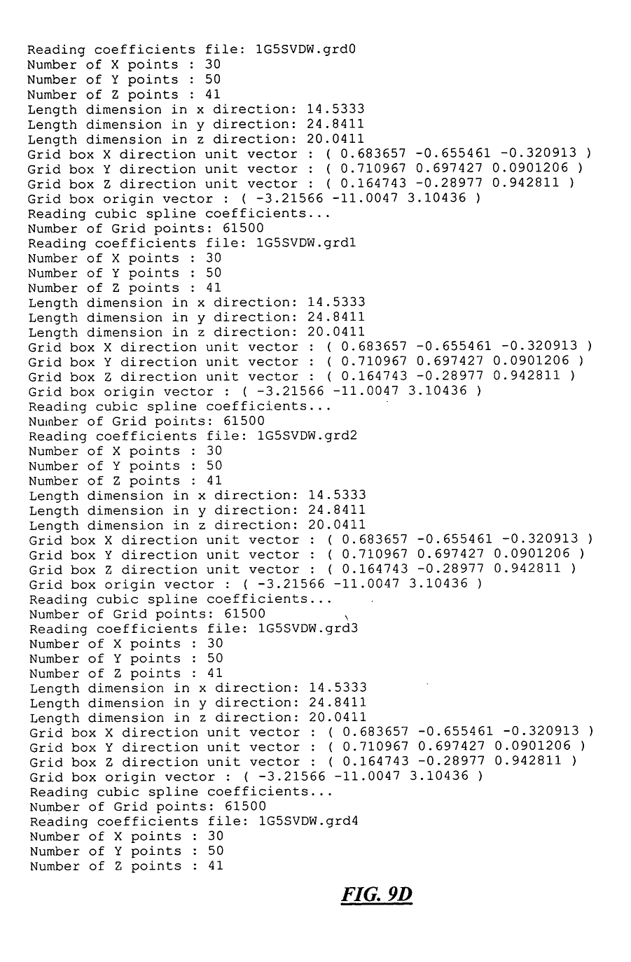

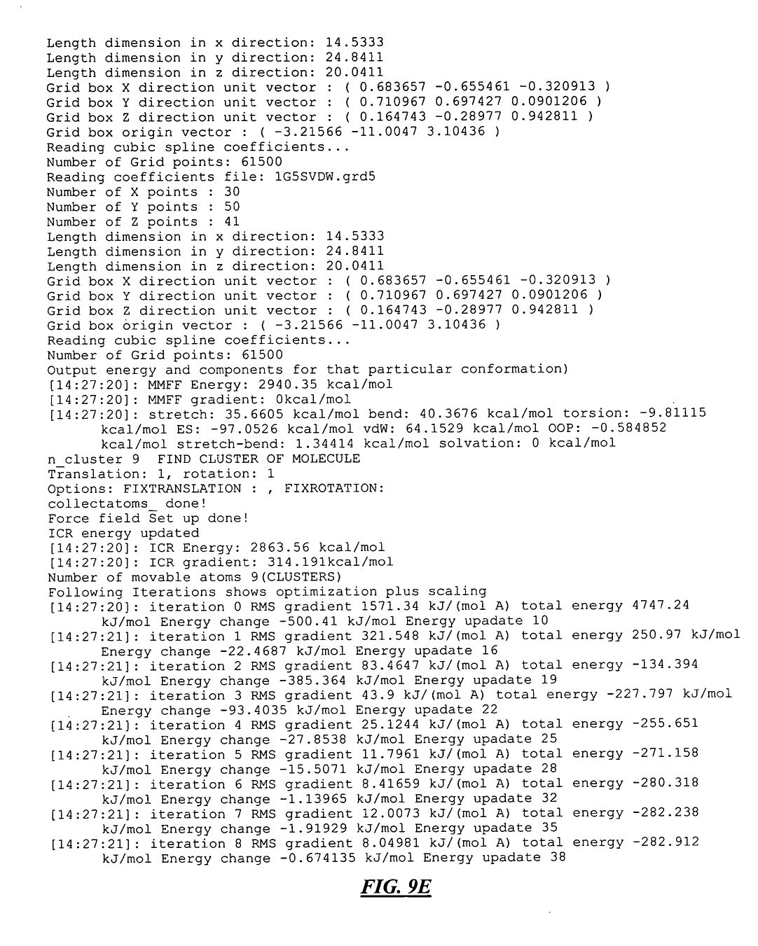

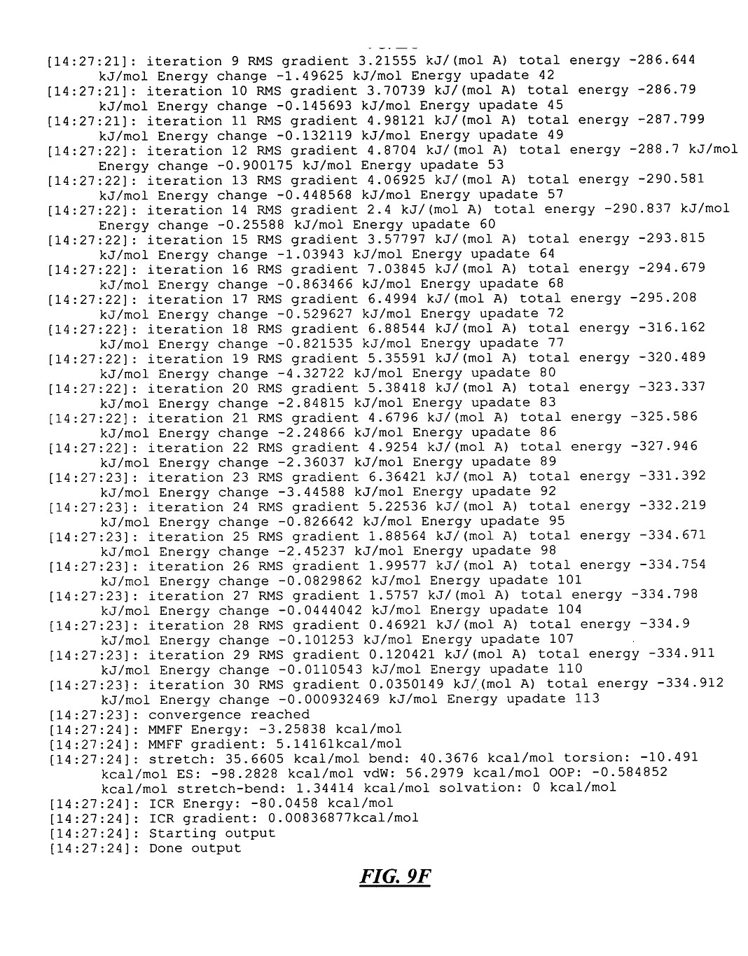

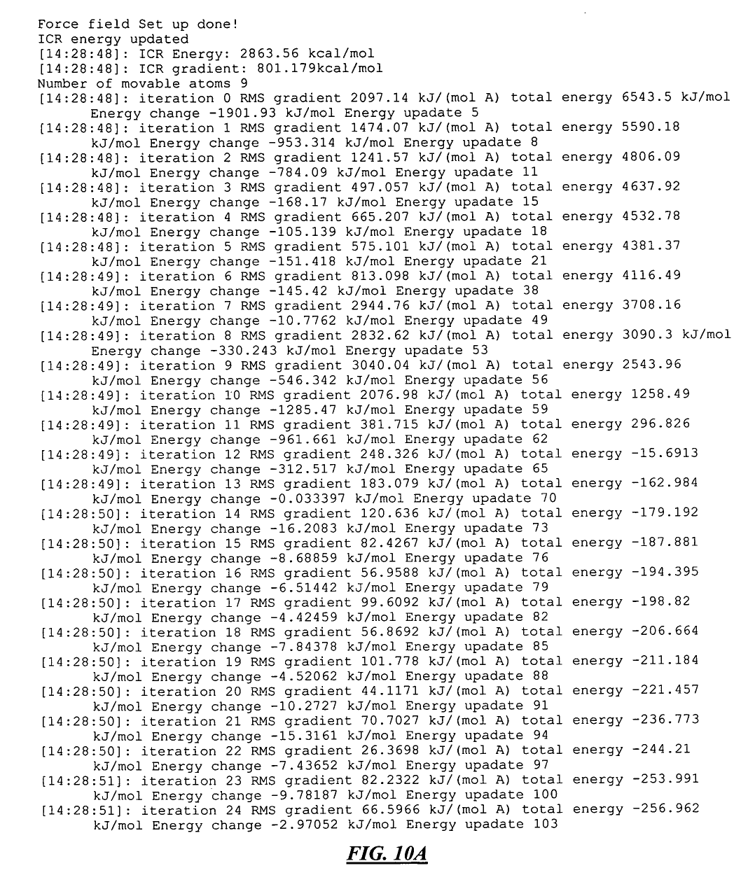

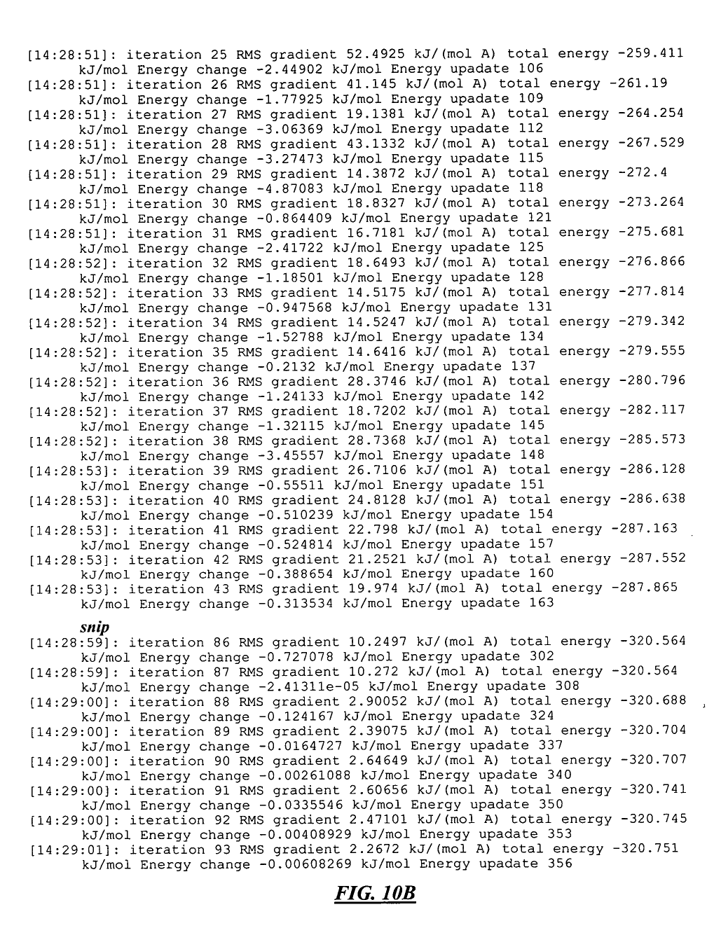

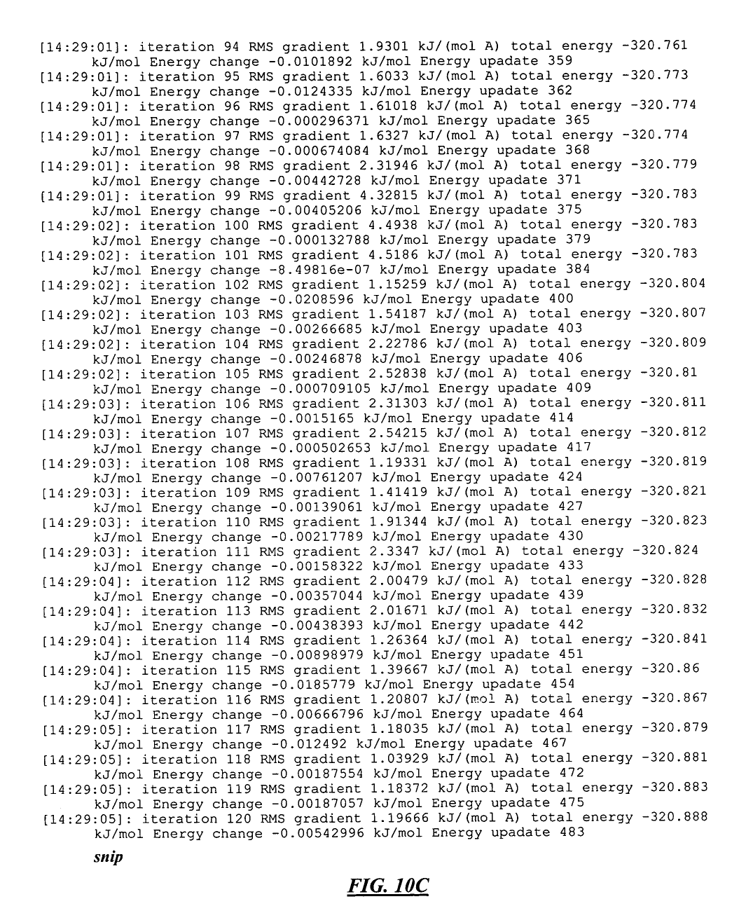

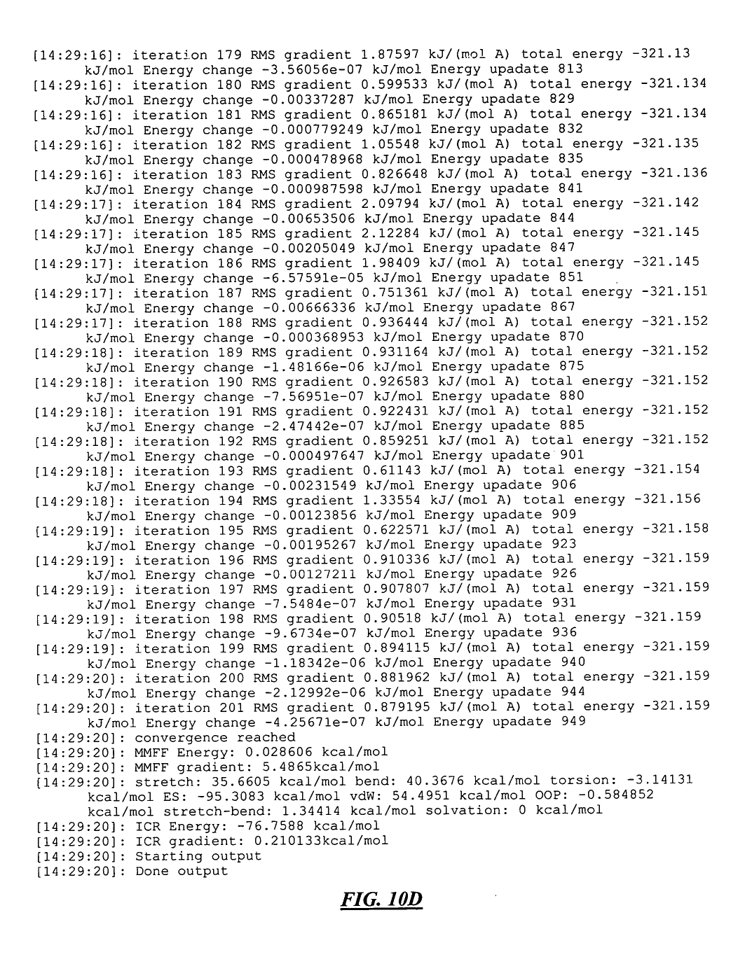

FIG. 9A-F shows portions of an example processing log file using resealing optimizations from a computer implemented method according to specific embodiments of the invention. FIG. 10A-D shows portions of an example processing log file using a non-rescaling optimizations from a computer implemented method according to specific embodiments of the invention. A comparison of these two log files shows that there are far fewer iterations in this example with a scaled method according to specific embodiments of the invention.

4. Embodiment in a Programmed Information Appliance

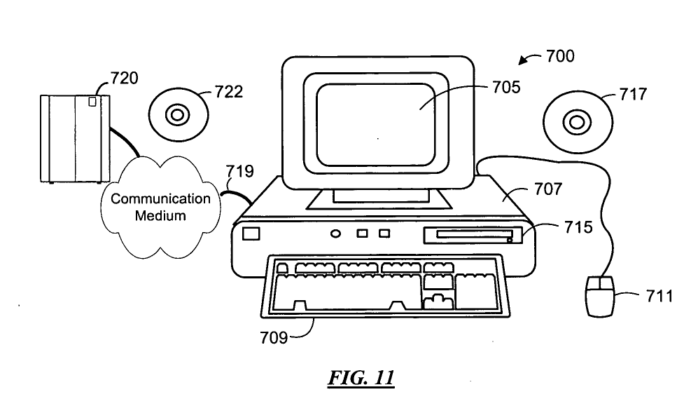

FIG. 11 is a block diagram showing a representative example logic device in which various aspects of the present invention may be embodied. As will be understood to practitioners in the art from the teachings provided herein, the invention can be implemented in hardware and/or software. In some embodiments of the invention, different aspects of the invention can be implemented in either client-side logic or server-side logic. As will be understood in the art, the invention or components thereof may be embodied in a fixed media program component containing logic instructions and/or data that when loaded into an appropriately configured computing device cause that device to perform according to the invention. As will be understood in the art, a fixed media containing logic instructions may be delivered to a user en a fixed media for physically loading into a user's computer or a fixed media containing logic instructions may reside on a remote server that a user accesses through a communication medium in order to download a program component.

FIG. 11 shows an information appliance (or digital device) 700 that may be understood as a logical apparatus that can read instructions from media 717 and/or network port 719, which can optionally be connected to server 720 having fixed media 722. Apparatus 700 can thereafter use those instructions to direct server or client logic, as understood in the art, to embody aspects of the invention. One type of logical apparatus that may embody the invention is a computer system as illustrated in 700, containing CPU 707, optional input devices 709 and 711, disk drives 715 and optional monitor 705. Fixed media 717, or fixed media 722 over port 719, may be used to program such a system and may represent a disk-type optical or magnetic media, magnetic tape, solid state dynamic or static memory, etc. In specific embodiments, the invention may be embodied in whole or in part as software recorded on this fixed media. Communication port 719 may also be used to initially receive instructions that are used to program such a system and may represent any type of communication connection.

The invention also may be embodied in whole or in part within the circuitry of an application specific integrated circuit (ASIC) or a programmable logic device (PLD). In such a case, the invention may be embodied in a computer understandable descriptor language, which may be used to create an ASIC, or PLD that operates as herein described.

5. Other Embodiments

The invention has now been described with reference to specific embodiments. Other embodiments will be apparent to those of skill in the art. In particular, a user digital information appliance has generally been illustrated as a personal computer. However, the digital computing device is meant to be any information appliance for performing numerical analysis and can include such devices as a personal digital assistant, scientific workstation, laboratory or manufacturing equipment, etc. It is understood that the examples and embodiments described herein are for illustrative purposes and that various modifications or changes in light thereof will be suggested by the teachings herein to persons skilled in the art and are to be included within the spirit and purview of this application and scope of the claims.

All publications, patents, and patent applications cited herein or filed with this application, including any references filed as part of an Information Disclosure Statement, are incorporated by reference in their entirety.

1. A method of predicting a conformation of a ligand inside a binding site using a computer system comprising:
representing a candidate ligand by topological clusters; and
scaling conformational and/or orientational degrees of freedom or their corresponding derivatives iteratively while applying one or more optimization and/or scoring and/or energy determination functions.

2. A method of predicting a conformation of a ligand inside a binding site using an information system comprising:
obtaining data indicating positions and types of atoms in a candidate ligand;
applying a clustering routine to create candidate ligand topological clusters, said clusters characterized by atoms that can be analyzed as non-rotating for portions of an analysis;
obtaining data indicating an energy environment and/or positions of a target conformation state; and
scaling conformational and/or orientational degrees of freedom iteratively while applying one or more standard optimization and/or scoring and/or energy determination functions.

3. The method according to claim 2 wherein said scaling further comprises:
scaling a gradient used to measure energy space; and
scaling one or more parameters used to sample ligand conformational space.

4. The method according to claim 2 further comprising:
adjusting inputs to one or more of said one or more standard optimization and/or scoring and/or energy determination functions based on a size of one or more of said clusters.

5. The method according to claim 2 further comprising:
adjusting torque inputs to one or more of said one or more standard optimization and/or scoring and/or energy determination functions based on size changes due to one or more child cluster rotations.

6. The method according to claim 2 further comprising:
using a rescaling factor that scales each cluster movement by its characteristic size;
wherein said rescaling factor rescales based on a radius of gyration about a cluster hinge axis; and
using a mathematically consistent transformation of scaled rotational degrees of freedom to allow using one or more optimization algorithms.

7. The method according to claim 2 further comprising:
outside of said one or more standard optimization and/or scoring and/or energy determination functions:
calculating a torque on a cluster based on an interaction function with other atoms in other clusters or molecules;
determining a radius of gyration (R_{g}) of the cluster, and
scaling the torque by the radius of gyration.

8. The method according to claim 7 further comprising:
wherein the radius of gyration (R_{g}) of a cluster is calculated from its moment of inertia (I) using I=M R_{g}² where M is the total mass of the cluster.

9. The method according to claim 7 further comprising:
after performing said calculating, said determining, and said scaling for a plurality of clusters of said ligand, inputting scaled torques to said one or more standard optimization and/or scoring and/or energy determination functions.

10. The method according to claim 7 further comprising:
wherein said optimization procedure outputs step sizes for one or more clusters;
using said step sizes to calculate a rotational angle of a cluster.

11. The method according to claim 10 further comprising:
recalculating a radius of gyration of a cluster when one of that clusters child clusters change position upon rotation.

12. The method according to claim 7 further wherein:
a torque of a cluster is calculated using Tᵢ(θ)=−∇_{θ}U, where i denotes the i^{th} cluster, θ is the rotation angle of the cluster about its hinge axis, U is an energy field; and ∇_{θ} is a gradient of the energy field with respect to the angle;

13. The method according to claim 12 further comprising:
scaling the torque for each cluster by its radius of gyration before passing to a gradient based optimization routines (e.g., Tᵢ^{new}(θᵢ)=Tᵢ(θᵢ)/Rᵢ, where Rᵢ is the radius of gyration of the i^{th} cluster).

14. The method according to claim 6 further comprising:
wherein said one or more optimization algorithms search for movements and/or step sizes (e.g., incremental rotation angles) for one or more clusters; and
probabilities of movement are weighted inversely proportional to corresponding radius of gyration.

15. A computer readable medium containing computer interpretable instructions that when loaded into an appropriately configuration information processing device will cause the device to operate in accordance with the method of claim 2.

16. An information processing system able to predicting conformation of a ligand inside a binding site comprising:
a first data store holding data indicating positions and types of atoms in a candidate ligand;
a logic processor able to apply a clustering routine to create candidate ligand topological clusters, said clusters characterized by atoms that can be analyzed as non-rotating for portions of an analysis;
a second data store holding data indicating an energy environment and/or positions of a target conformation state; and
said logic processor applying one or more logic modules able to scale conformational and/or orientational degrees of freedom iteratively while applying one or more standard optimization and/or scoring and/or energy determination functions.

17. A system for predicting conformation of a ligand inside a binding site comprising:
means for obtaining data indicating positions and types of atoms in a candidate ligand;
means for applying a clustering routine to create candidate ligand topological clusters, said clusters characterized by atoms that can be analyzed as non-rotating for portions of an analysis;
means for reading data indicating an energy environment and/or positions of a target conformation state; and
means for applying one or more logic modules able to scale conformational and/or orientational degrees of freedom iteratively while applying one or more standard optimization and/or scoring and/or energy determination functions.
